(19) United States
(12) Patent Application Publication
(10) Pub. No.: US 2023/0009992 A1
(43) Pub. Date: Jan. 12, 2023

(54) MESENCHYMAL STEM CELL CULTURE PRODUCT AND METHOD FOR PREPARING THE SAME

(71) Applicant: 3D GLOBAL BIOTECH INC., NEW TAIPEI CITY (TW)

(72) Inventors: Keng-Liang Ou, New Taipei City (TW); Hsu-An Pan, Yilan County (TW); Yi-Hsuan Kuo, Tainan City (TW)

(21) Appl. No.: 17/545,034

(22) Filed: Dec. 8, 2021

(30) Foreign Application Priority Data
Jul. 7, 2021 (TW) 110124906

(51) Int. Cl. C12N 5/0775

(52) U.S. Cl. CPC C12N 5/0666; C12N 5/0668; C12N 2501/119; C12N 2501/165; C12N 2501/15; C12N 2501/117; C12N 2501/105

(57) ABSTRACT

A mesenchymal stem cell culture product and a method for preparing the same are provided. The method for preparing the mesenchymal stem cell culture product includes the following steps. A predetermined quantity of mesenchymal stem cells is seeded in a flat culture device containing a first cell culture medium. When the mesenchymal stem cells proliferate to a target quantity, the first cell culture medium is replaced with a second cell culture medium, and the second cell culture medium is removed after incubation for 18 to 30 hours. A target cell culture medium is added and incubated for 48 to 72 hours. The target cell culture medium is collected repeatedly. The collected target cell culture medium is filtered and concentrated to obtain the mesenchymal stem cell culture product.

### CROSS-REFERENCE TO RELATED PATENT APPLICATION

This application claims the benefit of priority to Taiwan Patent Application No. 110124906, filed on Jul. 7, 2021. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a cell culture product, and more particularly to a mesenchymal stem cell culture product and a method for preparing the same.

### BACKGROUND OF THE DISCLOSURE

Stem cells are undifferentiated cells with the ability to differentiate and self-proliferate, and have been widely studied in modern society and used in regenerative medicine. Mesenchymal stem cells are adult stem cells, and are one of the stem cells that have been extensively studied in recent years. Mesenchymal stem cells can be isolated from various tissues in the human body (such as fat, teeth, the umbilical cord, and hair follicles), and can be used in repairing of injured tissues, anti-inflammation, transplantation, and so on. In recent years, studies have found that proteins secreted by the stem cells also have the effect of repairing cell functions. Therefore, how to extract a large amount of stem cell culture products is a major developmental trend in the related field.

Stem cell culturing can be divided into two-dimensional culture and three-dimensional culture. A traditional two-dimensional culture method is to allow cells to adhere and grow, such that a culture medium can be directly absorbed, and the cell activity is relatively stable. However, the capacity of the culture medium in a traditional culture dish is limited, and mass production cannot be performed. In the three-dimensional culture, the cells are allowed to be attached to a carrier and are cultured in a suspension state. Compared with the two-dimensional culture, a wider culture area is provided, and a large amount of the culture medium can be collected at once. However, in the three-dimensional culture, an adhesion effect of the cells will be affected by different materials of microcarriers, and a rotating flask or a fan-shaped fermentation tank is required to serve as a culture vessel. When the culture medium rotates, a shear force is generated and can cause cell pressure or cell damage. Moreover, when collecting the culture medium, the cells need to be further centrifuged before the culture medium can be collected. Therefore, the equipment cost for the three-dimensional culture is high, and a manual operation process is time-consuming. In addition, the quality of the product is unstable due to poor cell conditions.

Therefore, how to improve preparation methods, so as to overcome the above-mentioned shortcomings, improve a yield amount of the stem cell culture products, and achieve a stable quality, has become one of the important issues to be addressed in the relevant field.

### SUMMARY OF THE DISCLOSURE

In response to the above-referenced technical inadequacies, the present disclosure provides a mesenchymal stem cell culture product and a method for preparing the same, so as to achieve stability in production quantity and product quality, and in addition, enabling a culture medium to be repeatedly collected, so as to achieve a high level of utilization.

In one aspect, the present disclosure provides a method for preparing a mesenchymal stem cell culture product, which includes the following steps. In step (a), a predetermined quantity of mesenchymal stem cells is seeded in a flat culture device containing a first cell culture medium. In step (b), when the mesenchymal stem cells proliferate to a target quantity, the first cell culture medium is replaced with a second cell culture medium, and the second cell culture medium is removed after incubation for 18 to 30 hours. In step (c), a target cell culture medium is added and incubated for 48 to 72 hours. In step (d), the target cell culture medium is collected. In step (e), the step (c) to the step (d) are repeated for 1 to 5 times. In step (f), the collected target cell culture medium is filtered and concentrated, so as to obtain the mesenchymal stem cell culture product.

In certain embodiments, a source of the mesenchymal stem cells is hair follicles or skin.

In certain embodiments, the predetermined quantity is 1×10⁷ to 5×10⁷ cells.

In certain embodiments, a first cell culture medium includes a fetal bovine serum and an antibiotic, and the second cell culture medium does not include the fetal bovine serum and the antibiotic.

In certain embodiments, a cell attachment surface area of the flat culture device is 6000 cm² to 6500 cm².

In certain embodiments, the flat culture device includes 10 culture platforms, and each of the culture platforms has a surface area of from 600 cm² to 650 cm².

In certain embodiments, the target quantity is 6×10⁷ to 1×10⁸ cells.

In certain embodiments, in the step (f), the mesenchymal stem cell culture product is obtained by passing through a filter membrane and then through a tangential flow filtration system. A pore size of the filter membrane is 0.22 microns.

In certain embodiments, the step (f) further includes: using the tangential flow filtration system with a hollow fiber membrane having a molecular weight cut-off of from 3 kD to 5 kD to concentrate and filter the mesenchymal stem cell culture product in the target cell culture medium, and placing the mesenchymal stem cell culture product in a protein preservation solution.

In another aspect, the present disclosure provides a mesenchymal stem cell culture product, prepared by the method described above.

In certain embodiments, the mesenchymal stem cell culture product includes at least one selected from the group consisting of fibroblast growth factor (FGF), vascular endothelial growth factor (VEGF), transforming growth factor-θ (TGF-β), keratinocyte growth factor (KGF-2), and insulin-like growth factor (IGFs).

Therefore, in the mesenchymal stem cell culture product and the method for preparing the same provided by the present disclosure, by virtue of “seeding a predetermined quantity of mesenchymal stem cells in a flat culture device containing a first cell culture medium for cell proliferation”, “adding and incubating a target cell culture medium for 48 to 72 hours, and collecting the target cell culture medium”, and “filtering and concentrating the target cell culture medium, so as to obtain the mesenchymal stem cell culture product,” a large number of culture products with a stable quality can be obtained from the same manufacturing batch, thereby reducing the cost of stem cell culture and burden on relevant personnel.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a flowchart showing steps from a method for preparing a mesenchymal stem cell culture product according to the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

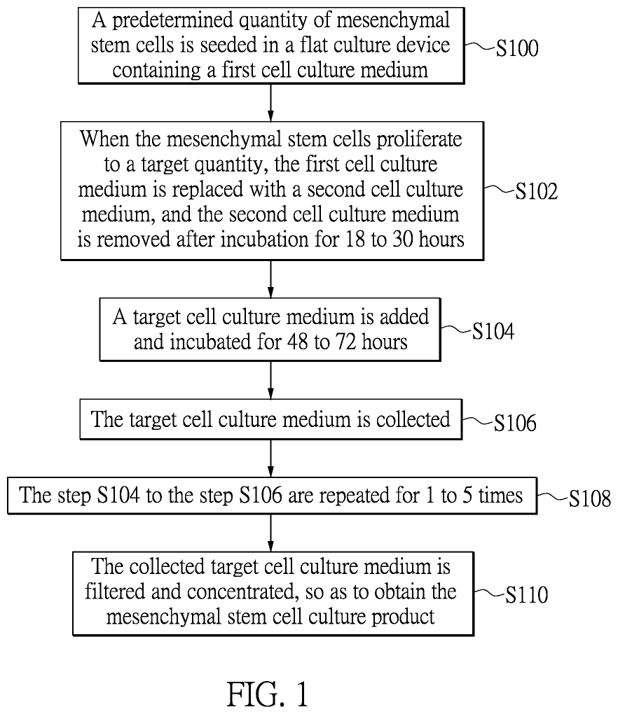

Referring to FIG. 1, the present disclosure provides a method for preparing mesenchymal stem cell culture product, which includes step S100 to S110. In step S100, a predetermined quantity of mesenchymal stem cells is seeded in a flat culture device containing a first cell culture medium. In step S102, when the mesenchymal stem cells proliferate to a target quantity, the first cell culture medium is replaced with a second cell culture medium, and the second cell culture medium is removed after incubation for 18 to 30 hours. In step S104, a target cell culture medium is added and incubated for 48 to 72 hours. In step S106, the target cell culture medium is collected. In step S108, the step S104 to the step S106 are repeated for 1 to 5 times. In step S110, the collected target cell culture medium is filtered and concentrated, so as to obtain the mesenchymal stem cell culture product. It should be noted that the method of the present disclosure utilizes the characteristics of the mesenchymal stem cells being adherent cells to culture the mesenchymal stem cells in a large-area cell culture device, so as to maintain cell integrity of the mesenchymal stem cells. Compared with cell culture in fermentation tanks, two-dimensional cell culture in a large area can reduce cell damage, reduce cell pressure, and provide a stable production environment for stem cells to secrete exosomes or growth factors. Accordingly, the quality stability of the mesenchymal stem cell culture product produced between batches can be maintained more easily.

In the step S100, the predetermined quantity of the mesenchymal stem cells is seeded in the flat culture device containing the first cell culture medium. A source of the mesenchymal stem cells of the present disclosure can be hair follicles or skin, and is preferably human hair follicle-mesenchymal stem cells (hHF-MSC). However, the present disclosure is not limited thereto.

In this embodiment, the predetermined quantity of the mesenchymal stem cells can be 1×10⁷ to 5×10⁷ cells, and is preferably 2×10⁷ to 3×10⁷ cells. In this embodiment, the target quantity of the mesenchymal stem cells can be 6×10⁷ to 1×10⁸ cells, and is preferably 7×10⁷ to 9×10⁷ cells. It is worth noting that the two-dimensional cell culture can generally only carry a small quantity of cells (for instance, about 8×10⁵ to 1×10⁶ cells in a 10-cm cell culture dish). When a large amount of cell culture products is to be collected, multiple collections and repetition of the steps of seeding the cells need to be performed, resulting in excessive operation time and burden on operators. The flat culture device of the present disclosure can be a multi-layer co-culture dish or a multi-layer platform culture device, but is not limited thereto. A surface area provided by the flat culture device for the attachment of the mesenchymal stem cells can be 6000 cm² to 6500 cm².

Specifically speaking, the flat culture device may further include 10 culture platforms. Each of the culture platforms has a surface area of 600 cm² to 650 cm², and the culture platforms are stacked and connected to each other. Therefore, in operation, the operator can efficiently incubate a large quantity of the mesenchymal stem cells in the same flat culture device, thereby shortening the operation time and ensuring that the cells are in a consistent state. In addition, a large amount of the culture medium can be collected at one time, which effectively reduces the risk of contamination caused by the multiple collections.

In this embodiment, the first cell culture medium can be high glucose Dulbecco's Modified Eagle Medium (DMEM-HG), Dulbecco's Modified Eagle Medium/Nutrient Mixture F-12 Ham (DMEM/F12, 1:1 mixture), or Dulbecco's Modified Eagle Medium/Nutrient Mixture F-12 Ham (DMEM/F12, 3:1 mixture), along with an additional 2.25 g/L glucose. It is worth noting that the first cell culture medium can further include a fetal bovine serum (FBS) and/or an antibiotic. The antibiotic may be a penicillin-streptomycin solution. For instance, the first cell culture medium can be a mixture of the Dulbecco's Modified Eagle Medium/Nutrient Mixture F-12 Ham at a ratio of 3:1, the additional 2.25 g/L glucose, 10% of the fetal bovine serum, and 1% of the penicillin-streptomycin solution, but is not limited thereto. Specifically speaking, 1 L to 2 L of the first cell culture medium will be added into the flat culture device with a surface area of 6000 cm².

In the step S102, when the mesenchymal stem cells proliferate to the target quantity, the first cell culture medium is replaced with the second cell culture medium, and the second cell culture medium is removed after incubation for 18 to 30 hours. For instance, to carry out the step S100 to the step S102 may require 48 hours to 96 hours. In operation, a new first cell culture medium can be replaced every 48 hours, so as to ensure activity of the mesenchymal stem cells. When a quantity of the mesenchymal stem cells is increased to 70-90% of the surface area of the flat culture device (preferably 80%), the target quantity is reached. The target quantity is about 6×10⁷ to 1×10⁸ cells, and is preferably 7×10⁷ to 9×10⁷ cells.

In order to prevent exosomes and the growth factors secreted by the cells from being interfered by the fetal bovine serum or the antibiotic and thereby affecting the quality of final products, the fetal bovine serum and the antibiotic in the first culture medium must be removed. In this embodiment, the second cell culture medium can be high glucose Dulbecco's Modified Eagle Medium (DMEM-HG), Dulbecco's Modified Eagle Medium/Nutrient Mixture F-12 Ham (DMEM/F12, 1:1 mixture), or Dulbecco's Modified Eagle Medium/Nutrient Mixture F-12 Ham (DMEM/F12, 3:1 mixture), along with an additional 2.25 g/L glucose. For instance, the second cell culture medium can be a mixture of the Dulbecco's Modified Eagle Medium/Nutrient Mixture F-12 Ham at a ratio of 3:1 and the additional 2.25 g/L glucose. It should be noted that the second cell culture medium does not contain the FBS or the antibiotic. In this step, the second cell culture medium is used for cell culture for 18 to 30 hours (preferably 24 hours), so as to ensure that the mesenchymal stem cells can metabolize the fetal bovine serum and/or the antibiotic that is originally contained in the first cell culture medium.

In the step S104, the target cell culture medium is added and incubated for 48 to 72 hours. In this embodiment, components of the target cell culture medium can be the same as the second cell culture medium. That is, the target cell culture medium does not contain the fetal bovine serum and/or the antibiotic. In this step, the purpose is to allow the mesenchymal stem cells to secrete the growth factors and the exosomes into the target cell culture medium, and the target cell culture medium is about 2 L. Therefore, after about 48 hours to 72 hours (preferably 48 hours), the step S106 is carried out to collect the target cell culture medium. Further, the step S104 to the step S106 are repeated for about 1 to 5 times (i.e., the step S108), and are preferably repeated for 3 to 5 times. About 10 L of the target cell culture medium can be collected. According to the step S104 to the step S108, the method of the present disclosure can collect a large amount of the target cell culture medium without centrifuging the cells. That is, a large amount of the target cell culture medium can be collected when the mesenchymal stem cells remain attached and are in a good growth condition.

In the step S110, the target cell culture medium is filtered and concentrated, so as to obtain the mesenchymal stem cell culture product. In operation, a 0.22-micron filter membrane is used to filter the target cell culture medium, so as to remove cell debris and impurities. Next, at a temperature of 4° C. to 8° C., a tangential flow filtration (TFF) system is used with a hollow fiber membrane having a molecular weight cut-off (MWCO) of 3 kD to 5 kD, so that proteins in the target cell culture medium are concentrated and filtered to obtain the mesenchymal stem cell culture product. Furthermore, the tangential flow filtration system can effectively reduce a filtration processing time and loss of the mesenchymal stem cell culture product, and can increase a concentration of the mesenchymal stem cell culture product. In some embodiments, a storage solution can be replaced during a concentration process, and the mesenchymal stem cell culture product (containing the growth factors and the proteins) in the target cell culture medium can be transferred to a protein preservation solution, so as to obtain the mesenchymal stem cell culture product. In this embodiment, the protein preservation solution can contain trehalose, mannitol, 0.01% of polysorbate 80 (commonly referred to as TWEEN® 80), and polyethylene glycol. It is worth noting that the mesenchymal stem cell culture product stored in the protein preservation solution has a better preservation effect.

The present disclosure also provides a mesenchymal stem cell culture product, which is prepared by the method described above. The mesenchymal stem cell culture product mainly contains proteins secreted by mesenchymal stem cells. For instance, the mesenchymal stem cell culture product includes at least one selected from the group consisting of fibroblast growth factor (FGF), vascular endothelial growth factor (VEGF), transforming growth factor-β (TGF-β), keratinocyte growth factor (KGF-2), and insulin-like growth factor (IGFs). However, the aforementioned examples describe only one of the embodiments of the present disclosure, and the present disclosure is not intended to be limited thereto.

### Beneficial Effects of the Embodiment

In conclusion, in the mesenchymal stem cell culture product and the method for preparing the same provided by the present disclosure, by virtue of “seeding a predetermined quantity of mesenchymal stem cells in a flat culture device containing a first cell culture medium for cell proliferation”, “adding and incubating a target cell culture medium for 48 to 72 hours, and collecting the target cell culture medium”, and “filtering and concentrating the collected target cell culture medium, so as to obtain the mesenchymal stem cell culture product,” a large number of culture products with a stable quality can be obtained from the same manufacturing batch, thereby reducing the cost of stem cell culture and burden on relevant personnel.

Furthermore, by incubating the cells in a co-layered culture device with a large area, the mesenchymal stem cells can be attached during growth, and there is no need for further centrifugation to obtain the culture medium. In this way, cell damage of the mesenchymal stem cells can be reduced. Through the technical characteristics of the present disclosure, the operator can effectively save the operation time and cost, and can effectively obtain a large batch of the mesenchymal stem cell culture product.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for preparing a mesenchymal stem cell culture product, comprising:
(a) seeding a predetermined quantity of mesenchymal stem cells in a flat culture device containing a first cell culture medium for cell proliferation;
(b) when the mesenchymal stem cells proliferate to a target quantity, replacing the first cell culture medium with a second cell culture medium, and removing the second cell culture medium after incubation for 18 to 30 hours;
(c) adding and incubating a target cell culture medium for 48 to 72 hours;
(d) collecting the target cell culture medium;
(e) repeating step (c) to step (d) for 1 to 5 times; and
(f) filtering and concentrating the collected target cell culture medium, so as to obtain the mesenchymal stem cell culture product.

2. The method according to claim 1, wherein a source of the mesenchymal stem cells is hair follicles or skin.

3. The method according to claim 1, wherein the predetermined quantity is 1×10⁷ to 5×10⁷ cells.

4. The method according to claim 1, wherein the first cell culture medium includes a fetal bovine serum and an antibiotic, and the second cell culture medium does not include the fetal bovine serum and the antibiotic.

5. The method according to claim 1, wherein a cell attachment surface area of the flat culture device is 6000 cm² to 6500 cm².

6. The method according to claim 5, wherein the flat culture device includes 10 culture platforms, and each of the culture platforms has a surface area of from 600 cm² to 650 cm².

7. The method according to claim 1, wherein the target quantity is 6×10⁷ to 1×10⁸ cells.

8. The method according to claim 1, wherein in the step (f), the mesenchymal stem cell culture product is obtained by passing through a filter membrane and then through a tangential flow filtration system; wherein a pore size of the filter membrane is 0.22 microns.

9. The method according to claim 8, wherein the step (f) further includes:
using the tangential flow filtration system with a hollow fiber membrane having a molecular weight cut-off of from 3 kD to 5 kD to concentrate and filter the mesenchymal stem cell culture product in the target cell culture medium, and placing the mesenchymal stem cell culture product in a protein preservation solution.

10. A mesenchymal stem cell culture product prepared by the method as claimed in claim 1.

11. The mesenchymal stem cell culture product according to claim 10, wherein the mesenchymal stem cell culture product includes at least one selected from the group consisting of fibroblast growth factor, vascular endothelial growth factor, transforming growth factor-β, keratinocyte growth factor, and insulin-like growth factor.
